(19) United States
(12) Patent Application Publication
(10) Pub. No.: US 2012/0132104 A1
(43) Pub. Date: May 31, 2012

(54) DENTAL MATERIALS CONTAINING ANTIMICROBIAL AGENTS, PARTICULARLY FOR THE PREVENTION OF PLAQUE DEPOSITS

(75) Inventors: Klaus Ruppert, Maintal (DE); Mario Beyer, Bad Homburg (DE); Sebastian Vogt, Erfurt (DE)

(73) Assignee: HERAEUS KULZER GMBH, Hanau (DE)

(21) Appl. No.: 13/382,699

(22) PCT Filed: Jul. 24, 2010
(86) PCT No.: PCT/EP2010/004551

§ 371 Date: Jan. 6, 2012

(30) Foreign Application Priority Data
Aug. 4, 2009 (DE) 10 2009 035 970.2

(51) Int. Cl. A61K 6/00; C07D 401/06; C08F 220/14; A61K 6/083; C08F 236/20

(52) U.S. Cl. 106/15.05; 523/115; 523/118; 525/327.3; 546/14

(57) ABSTRACT

Dental material is provided with at least one antimicrobial active substance that is applied onto inorganic particles or organic polymer beads and is bound non-covalently to the particles or polymer beads.

This results in a dental material on which plaque colonisation is prevented.

The invention relates to dental materials provided with antimicrobial properties, in particular for preventing the adhesion of plaque.

Polymeric dental materials, in particular those based on acrylate/methacrylate, which are introduced into the oral cavity for permanent residence therein, tend to be susceptible to colonisation by plaque at the surface of the material upon deficient oral hygiene.

Plaque consists of various bacteria and yeasts which become anchored firmly on surfaces such as, e.g., teeth or dental materials, through proteins and carbohydrates. More bacteria can then adhere to said first bacterial layer and thus form a three-dimensional colony. Certain substances that are released by the bacteria render this “biofilm” virtually impregnable by antibiotics. Aside from the aspect of hygiene, plaque, in its advanced state, leads to strong discolouration with ensuing negative aesthetic consequences.

### PRIOR ART

There are various conceivable options for reducing the colonisation of dental materials by plaque: Use of microbicidal agents, a protein-repelling surface based, e.g., on poly(ethylene glycols) or hydrophobic coating of the dental materials that renders the adhesion of bacteria on the material more difficult.

The use of quaternary ammonium salts as antimicrobial additives has been known for a long time. Accordingly, e.g. a silane having quaternary ammonium groups as functional group is produced by Microbeshield and marketed for providing filters, textile materials and wound dressings with antimicrobial properties. GB 1433303 A describes filling compound particles for plastic materials that are silanised and coated with quaternary ammonium salts. Diatomaceous earths or pyrogenic silicic acids treated in this manner are proposed, for example, for use in wood coatIngs, sealing compounds, catheters or textile fibres.

Moreover, additives based on cationic oligomers (Akacid®, made by PoC) and silver-containing additives [silver-containing glasses, salts, zeolites (U.S. Pat. No. 6,436,422 B1)] are known. Applying coatings of this type to filling compound particles in the nanometre size range, as are common for dental work, results in material with low abrasion stability with respect to natural mastication processes during the ingestion of food. Therefore, dental varnishes or fissure sealants provided with properties as described have to be applied repeatedly or are not generally suitable as protein-repelling layers. Using micro- or nanometre-sized metallic silver, natural colouring of gingiva imitations, fillings, veneering and/or artificial tooth material, as such is not attained. The silver-containing materials are susceptible to yellowish to grey colouring dependent on the grain size of the noble metal used.

JP 10025218 A describes inorganic filling active substances that are coated with a polymer layer containing antimicrobial groups. The layer is produced through polymerisation of corresponding (meth)acrylate monomers bearing phosphonium or quaternary ammonium groups.

According to DE 10 2005 042 078 A1, dental filling bodies are coated with polysaccharides with antimicrobial effect. Filling bodies coated this way with a polysaccharide are then enveloped in another polymer (page 4, paragraph 0034). In addition, a CC double bond is introduced into the polysaccharide, chitosan, in order to enable its integration into the polymer through polymerisation (page 4, paragraph 0037). The chitosan is then bound accordingly on the surface of the filling body (page 5, paragraph 0047).

### OBJECT OF THE INVENTION

It is the object of the present invention to provide a method that can be used to permanently prevent and/or delay the colonisation of dental materials by plaque without any adverse effect on the product properties of the dental material.

In this context, the core requirements, of which several need to be met, are as follows:
Homogeneous distribution of the active substance in the bulk material and on the surface of the material should be ensured, i.e. no uneven distribution just in spots.
The material must not comprise micro- or macro-sized pores after release of the active substance. This is important not only for aesthetic reasons, but also because any form ation of fissures is a starting point for re-colonisation by plaque.
It should be difficult to inactivate the active substance on the surface. This is expediently attained by replenishment of the active substance from the bulk material through diffusion.
A broad action spectrum against typical oral pathogens (bactericidal both for grampositive and gram-negative pathogens);
additional fungicidal properties, if applicable.
The active substance should be released in delayed manner.
The active substance should be colour- and odourless.
The release rate of the active substance should be sufficiently high for antimicrobial efficacy to be evident without manifestation of toxic or irritative/sensitising effects.
The active substance must not interfere with the polymerisation of the product and have no adverse effect on the properties of the material, there must be no phase separation (plasticiser effect).
The active substance must be associated with no more than a low probability of resistance formation by the typical oral pathogens.
No chemical reactions with the monomers, filling compounds, initiators, stabilisers and dyes must occur during storage and curing reaction.

The object is met by compositions according to claim 1. Further embodiments are evident from dependent claims 2-14.

Preferred active substances are those from: Majic-Todt, Ante, Dissertation 2003, “Prüfung von Lavasept® auf antiseptische Aktivität and Plaquehemmung (Test of Lavasept® for antiseptic activity and inhibition of plaque)”

| Active substance group | Antiseptic agents |
|---|---|
| Halogens | PVP iodine, sodium hypochlorite, |
| | tosylchloramide-sodium (chloramine T) |
| Guanidines | Chlorhexidine, alexidine |
| Cation-active compounds | Cetylpyridinium chloride |
| Quarternary ammonium | Benzalkonium chloride |
| compounds | |
| Pyrimidines | Hexetidine |
| Bispyridines | Octenidine dihydrochloride |
| Diphenylethers | Triclosan |

The compositions according to the invention are suitable for use in or as: filling composites, veneering composites, prosthetic materials, artificial teeth, elastic impression materials, protective varnishes, fissure sealants, dentine bonding materials and for hoof materials in veterinary medicine.

For production of the materials that are provided with antimicrobial properties according to the invention, it is expedient to dissolve the active substance in a suitable solvent, mix the solution with the inorganic filling compound and/or organic polymer and subsequently remove the solvent completely.

Monomers for radical polymerisation from the group of methacrylates and acrylates are mainly used in dental composites according to the invention:

Known viscous resins/monomers that are well-suited as dental material include polyurethane dimethacrylate (PUDMA), diurethane dimethacrylate (DUDMA) and/or polycarbonate dimethacrylate (PCDMA, condensation product of 2 parts hydroxyalkylmethacrylate and 1 part bis(chloroformate), as described in U.S. Pat. Nos. 5,276,068 and 5,444,104, moreover ethoxylated bisphenol A dimethacrylate (EBPDMA), as described in U.S. Pat. No. 6,013,694; and, in particular, Bis-GMA (Bowen monomer).

Diluent monomers are used to reduce the viscosity and to influence the wettability. Examples of suitable substances include hydroxyalkyl(meth)acrylates such as 2-hydroxy-ethyl(meth)acrylate, 2-hydroxypropyl(meth)acrylate and 4-hydroxybutyl(meth)acrylate; ethylene glycol unitscontaining (meth)acrylates such as ethylene glycol methacrylate, diethylene glycol methacrylate, triethylene glycol dimethacrylate and tetraethylene glycol dimethacrylate; dioldimethacrylates such as 1,4-butane-dioldi(meth)acrylate, dodecane-dioldi(meth)acrylate and 1,6-hexane-dioldi(meth)acrylate, and of these in particular 1,6-hexane-dioldimethacrylate (HDDMA). Further suitable monomers are, e.g., polyethylene glycol mono(meth)acrylate; glycerol di(meth)acrylate; trinnethylolpropane-di(meth)acrylat; pentaerythritol-tri(meth)acrylat; the (meth)acrylate of phenylglycidylether. Tri(ethylene glycol)dimethacrylate (TEGDMA) is particularly preferred.

The quantities of the diluent monomers and viscous resins can vary broadly and are in the range of approx. 1 to 70% by weight with respect to the dental material.

Cross-linkers are added, for example, in order to increase the final stability:

Suitable difunctional (meth)acrylate cross-linkers are mainly cross-linking bi- or multifunctional acrylates and/or methacrylates such as, e.g., bisphenol-A-di(meth)acrylate, bis-GMA (an addition product of methacrylic acid and bisphenol-A-diglycidylether), UDMA (an addition product of 2-hydroxyethylmethacrylate and 2,2,4-trimethylhexamethylene diisocyanate), di-, tri- or tetraethylene glycol di(meth)acrylate, trimethylolpropane tri(meth)acrylate, pentaerythritol tetra(meth)acrylate, and 1,4-butane dioldi(meth)acrylate, 1,10-decane dioldi(meth)acrylate or 1,12-dodecane dioldi(meth)acrylate.

It can be advantageous to add known monomers for ring-opening radical polymerisation showing little shrinkage, such as, e.g., mono or multifunctional vinylcyclopropanes and/or bicyclic cyclopropane acrylate derivatives (see DE 196 16 183 C2 and/or EP 03 022 855) or cyclic allylic sulfides (see U.S. Pat. No. 6,043,361 or U.S. Pat. No. 6,344,556), which can, in addition, also be used in combination with the above-mentioned di(meth)acrylate cross-linkers. Preferred monomers for ring-opening polymerisation include vinylcyclopropanes such as 1,1-di(ethoxycarbonyl)- or 1,1-di(methoxycarbonyl)-2-vinylcyclopropane or the esters of 1-ethoxycarbonyl- or 1-methoxycarbonyl-2-vinylcyclopropane carbonic acid and ethylene glycol, 1,1,1-trinnethylolpropane, 1,4-cyclohexanediol or resorcin. Preferred bicyclic cyclopropane derivatives are 2-(bicyclo[3.1.0]hex-1-yl)acrylic acid methyl or ethyl esters or the di-substitution products in 3-position thereof such as (3,3-bis(ethoxycarbonyl)bicyclo[3.1.0]hex-1-yl) acrylic acid methyl or ethyl esters. Preferred cyclic allyl sulfides are mainly the addition products of 2-(hydroxymethyl)-6-methylene-1,4-dithiepan or 7-hydroxy-3-methylene-1,5-dithiacylooctane and 2,2,4-trimethylhexamethylene-1,6-diisocyanate or the asymmetrical hexamethylene diisocyanate trimer, Desmodur® VP IS 2294 made by Bayer AG.

Also suitable are calix[n]arenes for cationic polymerisation according to general formula (I) as described in DE102007035734A1.

Known monomers for cationic ring-opening polymerisation showing little shrinkage include, e.g., glycidylethers or cycloaliphatic epoxides, cyclic ketene acetals, spiroorthocarbonates, oxetanes or bicyclic orthoesters.

Some examples are: 2-methylene-1,4,6-trioxaspiro[2.2]nonane, 3,9-dimethylene-1,5,7,11-tetraoxaspiro[5.5]undecane, 2-methylene-1,3-dioxepane, 2-phenyl-4-methylene-1,3-dioxolane, bisphenol-A-diglycidylether, 3,4-epoxy-cyclohexylmethyl-3,4-epoxycyclohexane carboxylate, bis(3,4-epoxycyclohexylmethyl)adipate, vinylcyclohexene dioxide, 3-ethyl-3-hydroxymethyloxetane, 1,10-decandiyl-bis-(oxymethylene)-bis-(3-ethyloxetane) or 3,3-(4-xylylene-dioxy)-bis-(methyl-3-ethyloxetane) and/or other epoxides specified in EP 0 879 257 B1. Suitable matrix systems for cationic polymerisation also include silicic acid polycondensates that bear the groups for cationic polymerisation, preferably, e.g., epoxide, oxetane or spiroorthoester groups, through hydrolytic condensation of silanes. Silicic acid polycondensates of this type are described, for example, in DE 41 33 494 C2 or U.S. Pat. No. 6,096,903.

The dental materials according to the invention that are based on monomers for radical polymerisation can be polymerised using the known radical initiators (see Encyclopedia of Polymer Science and Engineering, vol. 13, Wiley-Intersci. Pub., New York etc. 1988, 754 pp.).

Photoinitiators are particularly well-suited (see J. P. Fouassier, J. F. Rabek (eds.), Radiation Curing in Polymer Science and Technology, vol. II, Elsevier Applied Science, London and New York 1993) for the UV- or visible range, such as, e.g. benzoin ether, dialkyl benzilketals, dialkoxyacetophenones, acyl derivatives of the bisacylphosphine oxides, [alpha]-diketones such as 9,10-phenanthrene quinone, diacetyl, furil, anisil, 4,4′-dichlorobenzil and 4,4′-dialkoxybenzil and camphor quinone.

Moreover, azo compounds, such as 2,2′-azobis(isobutyronitrile) (AIBN) or azobis-(4-cyanovaleric acid), or peroxides, such as dibenzoylperoxide, dilauroylperoxide, tertbutylperoctoate, tert-butylperbenzoate or di-(tert-butyl)-peroxide, can also be used. Suitable initiators for hot setting are benzpinacol and 2,2′-dialkylbenzpinacols.

To accelerate the initiation by peroxides or [alpha]-diketones, it is common to use them in combination with aromatic amines. Time-proven redox systems include: combinations of benzoylperoxide or camphor quinone and amines, such as N,N-dimethyl-p-toluidine, N,N-dihydroxyethyl-p-toluidine, p-dimethylaminobenzoic acid ethylester or structurally related systems. Also well-suited are redox systems consisting of peroxides and reduction agents such as, e.g., ascorbic acid, barbiturates or sulfinic acids.

Dental materials according to the invention that are based on monomers for cationic polymerisation can be cured using the known cationic photoinitiators, in particular using diaryliodonium or triarylsulfonium salts, possibly in the presence of suitable sensitisers, such as, e.g., camphor quinone. Examples of suitable diaryliodonium salts that can be used together with camphor quinone or thioxanthonene as sensitiser in the visible range are the commercially available 4-octyloxy-phenyl-phenyl-iodoniumhexafluoroantimonate or isopropylphenyl-methylphenyliodoniumtetrakis(pentafluorophenyl)borate.

Moreover, dental materials according to the invention can contain one or more filling compounds, preferably organic or inorganic particulate filling compounds. Preferred particulate inorganic filling compounds are amorphous, spherical, nano-particulate filling compounds based on oxides, such as pyrogenic silicic acid or precipitated silicic acid, ZrO₂ and TiO₂ or mixed oxides of SiO₂, ZrO₂ and/or TiO₂ having a mean particle diameter of 10 to 200 nm, miniature-sized filling compounds, such as quartz, glass ceramic or glass powder having an average particle size from 0.2 to 5 [mu]m as well as radioopaque filling compounds, such as ytterbiumtrifluoride or nano-particulate tantalum(V) oxide or barium sulfate. Moreover, fibre-like filling compounds, such as glass fibres, polyamide fibres or carbon fibres, can be used as well.

And lastly, further additives can be added according to need to the dental materials according to the invention, such as, e.g., stabilisers, UV absorbers, dyes or pigments as well as solvents, such as, e.g., water, ethanol, acetone or ethyl acetate, or lubricants.

Accordingly, the dental materials according to the invention are preferably comprised of the following components depending on their intended use:

Cements according to the invention preferably contain: (a) 0.5 to 30% by weight, particularly preferably 0.5 to 20% by weight of at least one polymerisable calix[n]arene according to formula (I), (b) 0.01 to 2% by weight, particularly preferably 0.01 to 1.5% by weight initiator, (c) 1 to 30% by weight, particularly preferably 5 to 20% by weight of at least one further monomer for cationic and/or radical polymerisation and/or one further monomer for ring-opening polymerisation, preferably one multi-functional (meth)acrylate, (d) 5 to 70% by weight, particularly preferably 10 to 60% by weight filling compound, and (e) 0.01 to 5% by weight, preferably 0.01 to 2% by weight, particularly preferably 0.01 to 1% by weight additive, whereby the percentages specified add up to 100% in each case.

Filling composites according to the invention preferably contain: (a) 0.5 to 30% by weight, particularly preferably 0.5 to 20% by weight of at least one polymerisable calix[n]arene according to formula (I), (b) 0.01 to 5% by weight, particularly preferably 0.01 to 2% by weight, particularly preferably 0.01 to 1.5% by weight initiator, (c) 1 to 30% by weight, preferably 5 to 20% by weight, particularly preferably 5 to 15% by weight of at least one further monomer for cationic and/or radical polymerisation and/or at least one further monomer for ring-opening polymerisation, particularly preferably one multi-functional (meth)acrylate, (d) 5 to 85% by weight, particularly preferably 10 to 80% by weight filling compound, and (e) 0.01 to 5% by weight, preferably 0.01 to 3% by weight, particularly preferably 0.01 to 2% by weight additive, whereby the percentages specified add up to 100% in each case.

Coating materials according to the invention preferably contain: (a) 1 to 70% by weight, particularly preferably 1 to 50% by weight of at least one polymerisable calix[n]arene according to formula (I), (b) 0.01 to 5% by weight, preferably 0.01 to 2% by weight, particularly preferably 0.1 to 1.5% by weight initiator, (c) 5 to 70% by weight, preferably 5 to 60% by weight, particularly preferably 5 to 50% by weight of at least one further monomer for cationic and/or radical polymerisation and/or at least one further monomer for ring-opening polymerisation, particularly preferably at least one multi-functional (meth)acrylate, (d) 1 to 30% by weight, preferably 3 to 20% by weight, particularly preferably 3 to 15% by weight filling compound, preferably a nano-particulate filling compound, and (e) 0.01 to 5% by weight, preferably 0.01 to 3% by weight, particularly preferably 0.01 to 2% by weight, even more particularly preferably 0.01 to 1% by weight additive, (f) 0 to 70% by weight, particularly preferably 0 to 30% by weight solvent, whereby the percentages specified add up to 100% in each case.

Dental adhesives according to the invention preferably contain: (a) 0.5 to 50% by weight, particularly preferably 1.0 to 30% by weight of at least one polymerisable calix[n]arene according to formula (I), (b) 0.01 to 5% by weight, particularly preferably 0.01 to 2% by weight of at least one initiator, (c) 5 to 70% by weight, particularly preferably 5 to 60% by weight of at least one further monomer for cationic and/or radical polymerisation and/or at least one monomer for ring-opening polymerisation, particularly preferably at least one multi-functional (meth)acrylate, (d) 0 to 30% by weight, particularly preferably 3 to 20% by weight filling compound, and (e) 0.01 to 5% by weight, particularly preferably 0.01 to 3% by weight additive, (f) 0 to 50% by weight, particularly preferably 0 to 20% by weight solvent, whereby the percentages specified add up to 100% in each case.

Preferred prosthetic base materials according to the invention are based on methacrylates. Desired properties of a prosthetic base material are sought to be attained through, e.g., a high degree of cross-linking or the addition of impact strength-improving substances, such as polybutadiene polymers, as described in EP 1 702 633 A2. Prosthetic base materials usually contain pigments for production of a gingiva-like appearance.

The material used for artificial teeth is subject to very similar requirements as the filling materials described above and is therefore comprised of the same components.

Hoof materials are similar to prosthetic base materials in that they are two-component, selfcuring plastic materials, and they are used for repair of hoof damage in ungulate species (hoofed animals).

A material that is suitable for otoplasty is a material that is also used for dental elastic impression materials. This usually concerns addition- or condensation-cross-linking silicone materials, such as described, e.g., in EP 1 374 915 A2.

The invention is illustrated in more detail through the following examples. As is the case in the remaining description, specification of parts and percentages refer to the weight unless specified otherwise.

### EXAMPLE 1

### Providing Particles with Antimicrobial Properties

A total of 3 g octenidine dihydrochloride were dissolved in 97 g ethanol. A total of 8 g Aerosil OX 50 were added to the solution and the solution stirred vigorously at room temperature. Most of the solvent was removed by heating slightly to approx. 40-45° C. while continuing the stirring. The solvent was removed completely by heating gently at negative pressure (approx. 1-10 mbar).

### EXAMPLE 2

### Dental Materials containing Particles According to Example 1

Bis-GMA and TEDMA are used to generate a 70/30 mixture by slight heating and gentle stirring. Then the common photoinitiators and stabilisers as well as 65% by weight dental glass of a grain size of ˜1 μm were added. In order to set the rheology, 8% by weight pyrogenic silicic acid Aerosil OX50, treated with octenidine dihydrochloride were added. Moreover, colour pigments were added according to need to adjust the colour.

This resulted in a dental material that is provided with antimicrobial properties.

1. Dental material for providing antimicrobial properties to dental materials selected from the group consisting of composites, prosthetic base materials, adhesives, varnishes and sealants, comprising at least one antimicrobial active substance applied onto inorganic particles or organic polymer beads and adsorbed or bound to said particles or polymer beads through ionic interaction or van der Waals interaction selected from the group consisting of dipole-dipole, dipole-induced dipole and induced dipole-induced dipole interactions.

2. Dental material according to claim 1, wherein the inorganic particles are a dental filling compound.

3. Dental material according to claim 2, wherein the dental filling compound is selected from the group consisting of barium-aluminium-silicate glass, SiO₂, ZrO₂ and YbF₃.

4. Dental material according to claim 1, wherein said antimicrobial active substance is selected the group consisting of iminopyridinium derivatives, octenidine salts, dequalinium salts sanguarin and Akacid®.

5. Dental material according to claim 1, wherein the inorganic particles or organic polymer beads have a diameter of less than 50 μm.

6. Dental material according to claim 5, wherein the inorganic particles or organic polymer beads have a diameter of less than 10 μm.

7. Dental material according to claim 6, wherein the inorganic particles or organic polymer beads have a diameter of less than 2 μm.

8. Dental material according to claim 1, wherein the inorganic particles consist of SiO₂.

9. Dental material according to claim 1, wherein the inorganic particles consist of SiO₂ and one further heavy metal oxide.

10. Dental material according to claim 1, wherein the inorganic particles consist of a dental glass.

11. Dental material according to wherein said inorganic particles or organic polymer beads have a functionalized surface to promote solubilization.

12. Dental material according to claim 1, wherein the inorganic particles or organic polymer beads consist of PMMA or a methacrylate copolymer.

13. Dental material according to claim 1, wherein additives are present therein at up to 6% by weight.

14. Dental material according to claim 1, wherein one further antimicrobial component is homogeneously distributed in the dental material.

15. Dental material according to claim 14, whereby the further antimicrobial component is selected from the group consisting of monocationic antiseptic agents, dicationic antiseptic agents, oligo- or polymeric cationic antiseptic agents and antiseptic heavy metal compounds.

16. Dental material according to claim 1, wherein one further antimicrobial component is homogeneously distributed in the dental material, whereby the dental material can contain, in particular, monocationic antiseptic agents, dicationic antiseptic agents, oligo- or polymeric cationic antiseptic agents and antiseptic heavy metal compounds.

17. Dental material according to claim 9, wherein said heavy metal oxide is ZrO₂.
